(19) United States
(12) Patent Application Publication
(10) Pub. No.: US 2020/0229967 A1
(43) Pub. Date: Jul. 23, 2020

(54) WEIGHTED MEDICAL BLANKET FOR WARMING AND COOLING

(71) Applicant: Jill Drew, Lutherville, MD (US)

(72) Inventor: Jill Drew, Lutherville, MD (US)

(21) Appl. No.: 16/747,750

(22) Filed: Jan. 21, 2020

Related U.S. Application Data
(60) Provisional application No. 62/795,843, filed on Jan. 23, 2019.

(51) Int. Cl. A61F 7/00; A61F 7/02

(52) U.S. Cl. CPC A61F 7/0097; A61F 7/02; A61F 2007/0244; A61F 2007/0222; A61F 2007/0219

(57) ABSTRACT

A weighted medical blanket is provided for containing a polymer gel; the device of the invention maintains consistent weight over time and can be warmed or cooled to be applied to patients to provide heating or cooling and can be easily cleaned, reused, and its weight reconstituted through the insertion of water through an insertion valve and insertion sleeve suited to reduce flow out of the device. Disclosed is a method for alleviating post-operative shivering utilizing the device of the invention.

### RELATED APPLICATIONS

This application claims the benefit of U.S. Provisional Application No. 62/795,843 filed on Jan. 23, 2019.

### FIELD OF THE INVENTION

The present invention is directed toward a reusable weighted medical blanket with increased heat capacity to deliver warming or cooling to patients.

### BACKGROUND OF THE INVENTION

There are several situations that require the active external warming or cooling of patients in the course of medical evaluation and treatment. A common problem encountered in medical situations is mild hypothermia. Hypothermia can be caused by the administration of anesthesia and is common during and after surgical procedures. Core body heat redistributes to the skin surface through anesthetic-induced vasodilation and depression of hypothalamic thermoregulatory centers. Heat loss occurs mostly through skin via radiation and convective heat transfer.

After general anesthesia, shivering is a common complication in both normothermic and hypothermic patients. Postanesthetic shivering mainly contributes to patient discomfort and, moreover, also to morbidity by increasing oxygen demand and elevating intraocular or possibly intracranial pressure. Prevention or therapy of postanesthetic shivering includes the maintenance of normothermia, adequate pain therapy, and the administration of opioids, especially meperidine also known as the brand name Demerol. Administration of meperidine increases the risk of seizure.

Unrecognized and untreated intraoperative hypothermia remains a common avoidable scenario in the modern operating room. Failure to properly address this seemingly small aspect of the total operative care has been shown to have profound negative patient consequences including increased incidence of postoperative discomfort, surgical bleeding, requirement of allogenic blood transfusion, wound infections, and morbid cardiac events. All of these ultimately lead to longer hospitalizations and higher mortality. To avoid such problems, a device for warming a patient is needed as well as a method of using a warming device.

As many as fifty percent of trauma patients transported by EMS are unintentionally hypothermic by the time they reach the hospital. Hypothermic trauma patients are less likely to survive their injuries, when compared to similar patients who are normothermic. In other situations, a patient can be too warm and require cooling. Malignant hyperthermia is a condition characterized by hyperthermia, tachycardia, tachypnea, increased carbon dioxide production, increased oxygen consumption, acidosis, muscle rigidity, and rhabdomyolysis. This condition is triggered by general anesthetic gases, succinylcholine, and more rarely stress, heat, or exercise. Malignant hyperthermia occurs as a result of patient anesthesia at a rate of up to 0.02% of hospital patients.

Cardiac arrest outside the hospital kills roughly 250,000 Americans each year. Worldwide, the average survival rate for out-of-hospital cardiac arrest is just 6%. And, those who survive are at risk for neurologic injury. Historically, only about 20% of cardiac arrest survivors who remained comatose have awakened with a good neurologic outcome. Therapeutic hypothermia (including intentional cooling) holds out the promise of improving the rate of positive outcomes. It is therefore necessary to provide a reliable and portable device and method for heating or cooling a patient.

Current approaches to warming and cooling patients include the use of heated blankets, forced air warming devices, warmed IV fluids, warm/cool lavages and ice packs. According to the American Reusable Textile Association, 2 to 3 percent of a hospital's budget is spent on laundry and linen services. There has been a strong push to move outpatient surgery from hospital settings to Ambulatory Surgery Centers (ASCs). ASCs do not routinely possess linen facilities on site, and it is therefore necessary to contract with outside vendors. As with the hospital, the contracted laundry represents a large expense related to the overall budget.

Heated conventional blankets are the first line of defense against hypothermia. Typically, textile blankets are placed in blanket warmers prior to placement on patients. Conventional blankets are inefficient and have poor heat retention requiring multiple layers. In addition to poor heat conduction and retention, the use of multiple blankets have several collateral disadvantages, including increased cost, increased water consumption for cleaning, increased electricity consumption and use of chemicals necessary to comply with the standards for cleaning linen, risk of infection from soiling, and increased risk of musculoskeletal injury of staff from dragging and lifting heavy linen bags. Workers in the healthcare industry sustain 4.5 times more overexertion injuries than any other type of worker. Forced air patient warming devices are another method used to combat hypothermia. These systems, which require a constant source of electricity, have been implicated in increasing the risk of infection. Additionally, the use of a forced hot air system requires frequent reassessment to avoid overheating and potentially death. Warmed IV fluids and lavages require specially trained clinicians and may be contraindicated in some patients. In some cases IV narcotics are necessary to stop post-operative shivering.

Current methods to treat hyperthermia are equally cumbersome. Cooling water blankets require a constant electricity source and are often not available. Additionally, they may leak and create risk for slips and falls, as well as provide a medium for infection. Cold saline lavages require specially trained clinicians and are contraindicated in some patients. Foley catheter lavages increase risk of urinary tract infections, and require specially trained clinicians for placement. Wet sheets and towels increase the risk of infection and increase the risk of slips and falls from dripping on the floor. Spray bottles and fans are not effective in a life threatening hyperthermic emergency. Using ice bags during a malignant hyperthermia crisis is ineffective. Filling ice bags takes times and ties up limited staff resources needed for other emergency interventions.

What is needed is a cost effective, simple, and efficient method to warm and cool patients for an extended period of time without the need for excessive laundry, or a continuous electrical source, and avoids the use of opioids to stop or prevent post-operative shivering.

What is needed is a simple multi-use medical blanket that can be heated or cooled using existing blanket warmers or refrigerators; a device with a high heat capacity that can be easily heated or cooled, quickly deployed for use with a patient, and can also be readily returned to repeated service. The device could serve multiple purposes and occupy less space.

### SUMMARY OF THE INVENTION

It is an aspect of the present invention to provide a device with elevated heat capacity. It is a further aspect of the present invention to provide an embodiment providing a weighted medical blanket that is reusable, contains a substance that can be heated or cooled and retain flexibility, is suited for placement on a patient, and comprises a surface that is readily cleaned and returned to service to provide a reusable weighted blanket. It is a further object of the invention to provide a method for treating post-operative hypothermia by placing a weighted, heated device on a patient to alleviate shivering. It is an additional object of the invention to provide a device that can be heated or cooled using existing blanket warmers or refrigerators.

It is a further aspect of the present invention to provide a device that comprises a polymer gel that can be reconstituted, provides a lower initial weight, and provides longevity of use by allowing a user to add a liquid, for example water, to achieve a higher weight and to achieve or maintain a higher heat capacity. The objects of the invention can be attained with a reusable medical device comprising an inner polymer envelope comprising a first inner layer and a second inner layer surrounded by an outer polymer envelope comprising a first outer surface and a second outer surface where said inner polymer envelope containing a water absorbing polymer gel, and said outer polymer envelope is sealed with said inner polymer envelope to create at least a first edge and a second edge.

Further aspects of the invention can be achieved by an insertion valve that accepts one-way flow into the device and an insertion sleeve that facilitates delivery of solid or liquid into the device and comprises a check valve, or baffles, or a self-closing configuration to retain contents within the device. A method of treating a patient to alleviate post-operative shivering is disclosed comprising the steps of warming a weighted medical blanket of the invention and placing the device on a patient to provide both weight and heat.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

Further features and advantages of the present invention, as well as the structure and operation of various embodiments of the present invention, will become apparent and more readily appreciated from the following description of the embodiments, taken in conjunction with the accompanying drawings of which:

FIG. 1 is a top view of a device in an embodiment of the invention.

FIG. 2 is a bottom view of a device in an embodiment of the invention.

FIG. 3 is a sectional view of a device in an embodiment of the invention.

FIG. 4 is a perspective view of an injection valve and insertion sleeve in an embodiment of the invention.

FIG. 5 a view of a device as applied to a patient in a method of utilizing the invention.

FIG. 6 is a view of a device as applied to a patient in a method of utilizing the invention.

### DETAILED DESCRIPTION

Reference will now be made in detail to the presently preferred embodiments of the invention, examples of which are illustrated in the accompanying drawings, wherein like reference numerals refer to like elements throughout.

The present inventive concept relates to a weighted medical device that can be used as a blanket for heating or cooling a patient.

The current invention provides an alternative to current methods of heating and cooling patients suffering from hypothermia or hyperthermia. It has been found that a class of compounds provide high heat capacity as well as a weighted warming device. It has also been found that the use of a weighted heating device produces unexpected results in that post-operative shivering is quickly alleviated. An absorbent gel can be utilized with water to fill a medical device. The resulting device provides high heat capacity and mass greater than a conventional textile blanket.

The device can be joined with additional devices to provide a combined device large enough to accommodate patients of all sizes and in different positions. An example might be a person sitting up in a chair or perhaps trapped in a car prior to extrication. The medical blanket can be reused and wiped clean using disinfectants in between patient use, reducing risk of infection.

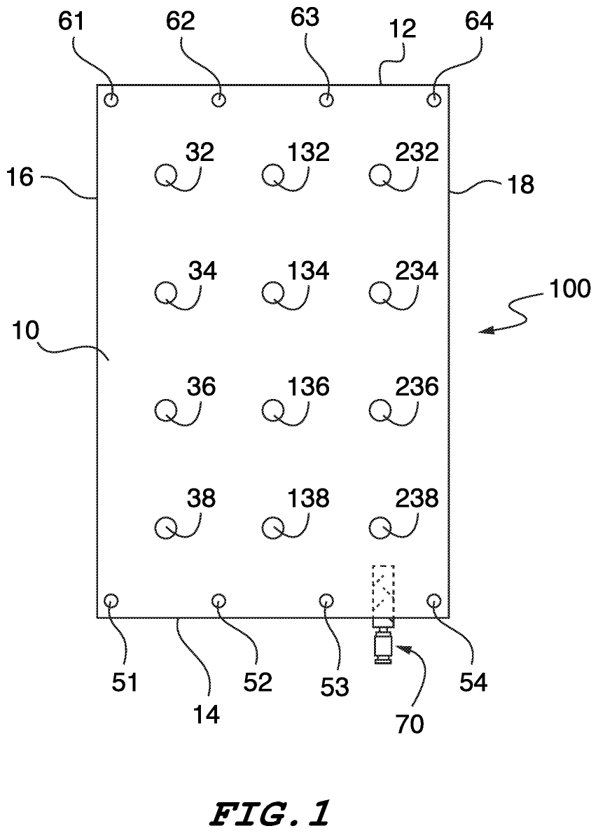

FIG. 1 presents a top view of a device in an embodiment of the invention. A weighted medical blanket 100 can be constructed with a vinyl shell backed with PVC to prevent porosity. In an embodiment, the blanket 100 can be filled with an absorbent polymer. Decreasing water evaporation through reduced porosity reduces water loss and weight loss of the blanket 100. The outside shell can comprise a first outer surface 10 and a second outer surface 20. The sides can be heat sealed to create an outer polymer envelope first edge 12 and second edge 14. In a particular configuration, third edge 16 and fourth edge 16 can be provided. First edge 12 can be configured with attachment elements for example attachment elements 61, 62, 63, and 64. Second edge 14 can be configured with attachment elements 51, 52, 53, and 54 for example snaps. These can be single sided attachment elements or double-sided attachment elements. Double sided attachment elements can attach to corresponding fasteners. Fasteners allow multiple devices to be joined together to provide different sizes and configurations for different needs. This is particularly helpful when a patient must be awkwardly positioned during surgical procedures, or as a result of trauma, e.g. a car accident.

First outer surface 10 can be attached by a method, for example heat welded, to second surface 20 at selected locations to reduce the movement of substances interior to the blanket 100. For example first weld 32, second weld 34, third weld 36, and fourth weld 38 can be located to create a resistance to flow within the blanket 100 and thereby decrease pooling and ensuring even distribution of the contents. A second set of welds including fifth weld 132, sixth weld 134, seventh weld 136, and eighth weld 138 can be positioned with a third set of welds including ninth weld 232, tenth weld 234, eleventh weld 236, and twelfth weld 238 to collectively restrain flow within the blanket. More or less welds can be provided as needed in different shapes and sizes of weighted medical blanket 100.

The inside of the weighted medical blanket 100 can be filled with a polymer gel, for example a polyacrylate gel. In an embodiment, a kilogram or more of a polymer gel 40 can be contained within blanket 100. One to two kilograms of water, or more, can be added to the blanket 100 to create a blanket weight of two to three kilograms or more. In an embodiment, one kilogram of polyacrylate gel can be combined with two kilograms of water to create a blanket 100 that weighs more than three kilograms and stays warm or cold outside of warmers or refrigerators for an hour or more. In order to provide additional weight a blanket 100 of the invention can be combined with another blanket 100 to provide additional weight. Therefore a 2 to 3 kg blanket can be snapped to a second 2 to 3 kg blanket to provide a combined device weighing 4 to 6 kg.

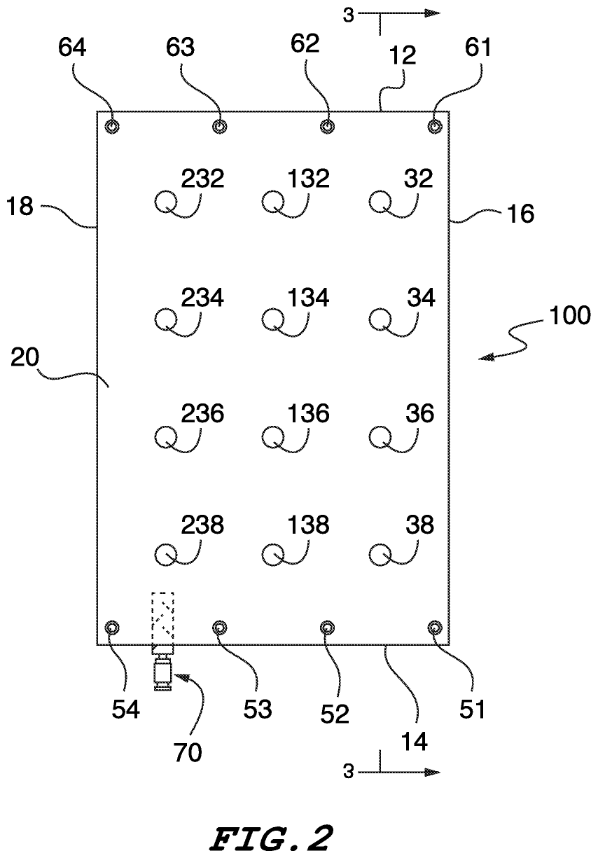

FIG. 2 presents a bottom view of a device in an embodiment of the invention. In an embodiment, first outer layer 10 and second outer layer 20 can comprise a thin liquid-impermeable flexible fabric, such as nylon or vinyl, or other polymer plastic.

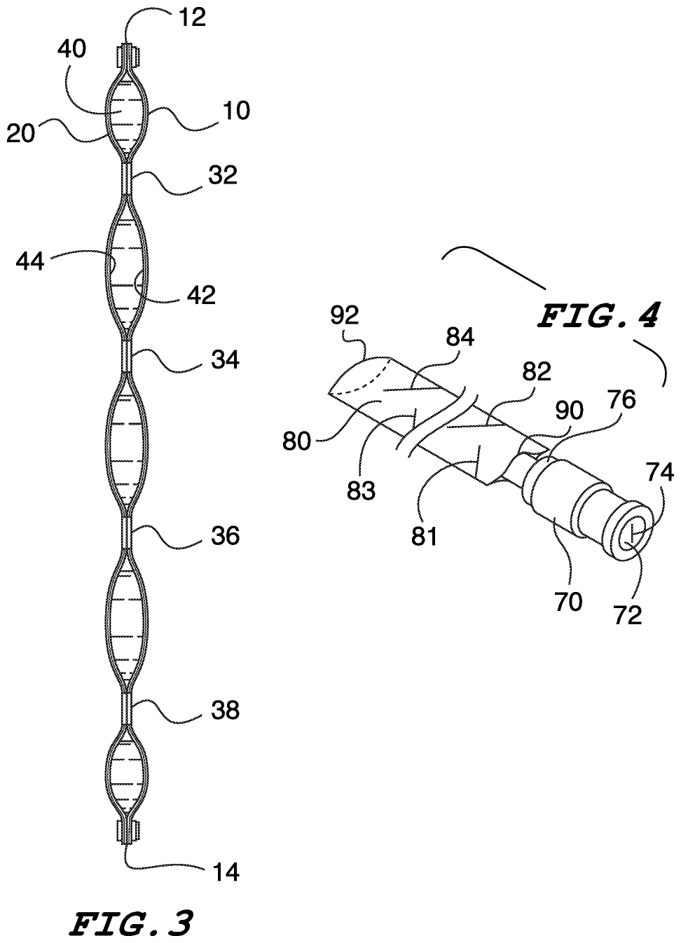

FIG. 3 presents a sectional view of a device in an embodiment of the invention. In order to reduce water loss from the device, blanket 100 can comprise dual layer construction. Dual layer construction decreases porosity of the device and the entrance of air or loss of water vapor. The effect is a reduction in the rate of water evaporation. First inner layer 42 and second inner layer 44 can comprise a polymer fabric, for example poly vinyl chloride, PVC. The inner and outer sheets can be sealed along their edges by thermal sealing or heat welding to form a leak-proof container or inner polymer envelope. Insertion valve 70 can be utilized for the addition of water to the device 100. The outer layers 10, 20 and the inner layers 42, 44 can be heat sealed together to reduce the flow of the contents of the device. In an embodiment, the four layers can be heat sealed together in a regular pattern to form welds, for example weld 32, which can comprise an octagonal shape. In an embodiment, the blanket 100 is generally planar with a rectangle-shape, and includes snaps on the top and bottom of the pad, useful for connecting more than one pad together, and for application on, for example, a patient's torso as shown in FIG. 5. However, the pad need not be planar and rectangular shaped and may comprise any shape and be non-planar to fit around a joint, for example, a patient's arm.

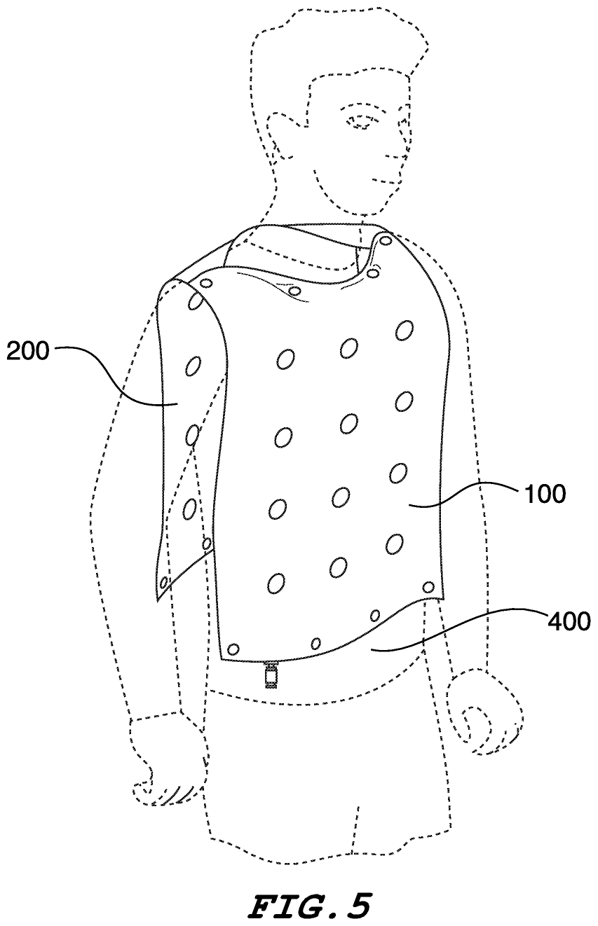

FIG. 4 presents a perspective view of insertion valve 70 that can be utilized to add water to the interior of the blanket 100. Insertion valve 70 can be novel or known in the art, for example a needleless insertion valve. Insertion valve 70 can be constructed of a clear PVC plastic that tunnels between first internal layer 42 and second internal layer 44 and allowing for the passage of powder and or water. The external opening of the insertion valve can be positioned flush with an edge, for example first edge 12. Insertion valve 70 provides access to the interior of the device for the insertion of liquid or powder or both and can be connected to insertion sleeve 80 to facilitate the insertion and retention of polymer gel or water. The distal end 92 of the insertion sleeve 80 passively opens with the pressure of inserting powder or liquid. When filling is complete, and there is no insertion pressure, the distal end 92 of the insertion sleeve 80 self-seals to prevent backflow of powder, water, gel, etc. Insertion sleeve 80 can be made of materials biased to lie flat and self-close. The insertion valve 70 can be used numerous times to achieve desired mass of the weighted blanket 100. The device of the invention can be constructed and sold without water to provide advantages including reduced shipping weight. Blanket 100 can be combined with inserted fluid, e.g. water prior to use.

To evaluate and counteract evaporation, the blanket can be weighed to determine water loss and a corresponding amount of water can be added to the device via insertion valve 70. Reduced evaporation is an object of the invention and material selection is aimed at increasing comfort and reducing water loss. When heated, water can evaporate and is desorbed in the vapor state. After complete desorption, the polymer particles resume their initial dry powder state causing the blanket 100 to flatten and become lighter. Insertion valve 70 can be provided with a check valve 72 that prevents leaks or backflow out of the blanket 100 through insertion valve 70. In an embodiment, check valve 72 can be a septum. Restoring water content to the device provides for extended use over a longer period of time and reducing replacement costs. In an embodiment, a needless syringe with a male luer connector can attach to a female luer 74 located on the insertion valve 70 of the blanket 100. Sleeve 80 can be connected with proximal end 90 to attachment means 76. In an embodiment, sleeve 80 can be embossed or welded with flow restrictions, e.g. first baffle 81, second baffle 82, third baffle 83, and fourth baffle 84 to further reduce flow of liquid out of the device, and maintain a flat or closed configuration of insertion sleeve 80. More or less baffles can be provided in the spirit of the invention. A user can be instructed to smooth the sleeve 80 flat in an interior direction to facilitate the movement of fluids into the device and the closure of sleeve 80.

A suitable absorbent polymer gel 40 includes polyacrylate gel. For example, sodium polyacrylate, also known as waterlock, is a sodium salt of polyacrylic acid with the chemical formula [—CH₂—CH(CO₂Na)—]ₙ. This superabsorbent polymer has the ability to absorb as much as 100 to 1000 times its mass in water. In addition to its water absorbing quality, polyacrylate gel is biodegradable and nontoxic rendering it suitable or use with patients. Other water absorbing polymers can be utilized in the spirit of the invention to provide polymer gel 40.

Insertion sleeve 80 can be attached to valve 70 via attachment means 76 by heat sealing, glue, or other mechanical connection such as reducing the diameter of the proximal end 90 of insertion sleeve 80.

It is necessary to maintain the consistent weight of the blanket as it is directly correlated with length of time for heat retention (45 minutes to 1 hour) and the ability to stop post-operative shivers without the use of opioids. Although there are studies to support the positive effects of weighted blankets on the nervous system, it has never been studied in the area of post anesthesia. It has been consistently observed by the inventor that post op shivering stops almost immediately with the application of the medical blanket. It is unclear if it is the weight, heat, or the two factors combined that contribute to the cessation of shivering. The present invention provides a method of treating post-operative shivering by applying a heated weighted blanket to a patient. The weight combined with heat activates the afferent nervous system, which sends massaging to the recently anesthetized brain that homeostasis has been reached. Afferent neurons are sensory neurons that carry nerve impulses from sensory stimuli towards the central nervous system and brain, while efferent neurons are motor neurons that carry neural impulses away from the central nervous system and towards muscles to cause movement.

The weighted medical blanket 10 can be placed in existing blanket warmers or refrigerators for a period of 30 minutes. After that time, the warmed device can be placed on the patient for the purpose of heating or cooling. Disclosed herein is a method for treating patients by applying a heated weighted medical blanket 100 on a patient to provide heating and avoid negative consequences of shock. Additionally, medical blanket 100 can be heated and then applied to a patient to arrest post-operative shivering following anesthesia. Medical blanket 100 can be cooled and then applied to a patient.

FIG. 5 presents a view of a patient 40 in an upright posture. In instances where a patient is injured in a car accident, but can not be immediately removed from the vehicle, weighted medical blanket 100 can be heated in advance by paramedics and applied to the patient to keep the patient warm while others provide access to the vehicle. A second weighted medical blanket 200 can be fastened to blanket 100 and applied over the head of the patient.

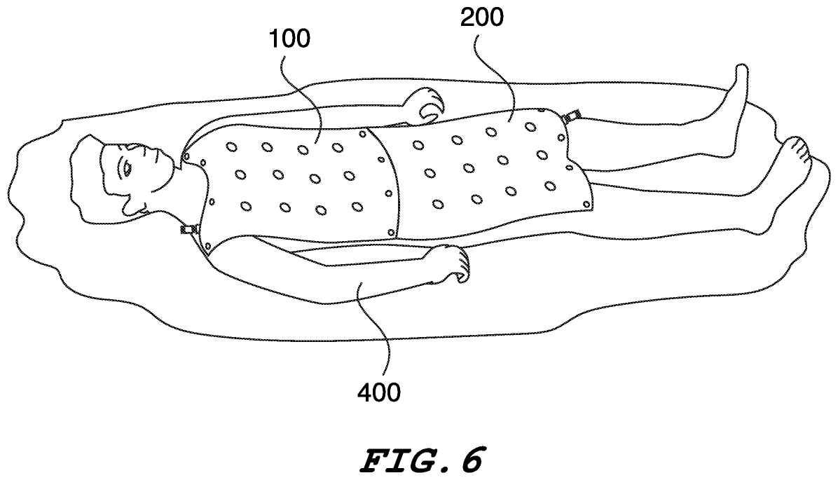

FIG. 6 presents a view of a patient 400 laying prone. Heated blanket 100 can be combined with a second heated blanket 200 to cover a significant portion of the patient. This can be applied prior to moving the patient and is compatible with a mobile bed or stretcher.

Weighted medical blanket 100 can be placed in a refrigerator set at a desired temperature for medical use. The water and polymer gel 40 provide heat capacity to provide a cooling function to a patient similar to the uses shown in FIGS. 5 and 6. When a patient is overheated, the same device, weighted medical blanket 100 can be provided from a commonly available refrigerator to provide a cooling function.

The invention provides multiple advantages. The device can be warmed and applied to a patient to arrest post-operative shivering without the use of narcotics. The device can be used with existing blanket warmers and refrigerators. The device effectively remedies hypothermia and hyperthermia. The device can provide heating or cooling for more than 45 minutes without the need for electricity. As shown in FIGS. 5 and 6, deploying a device at the scene of an accident may require standalone operation. The device can be combined by releasable attachments to provide cooling or heating over a large surface area. The weighted medical blanket 100 is reusable and significantly reduces use of linen and therefore water cost and laundry cost. Use of weighted medical blanket 100 reduces the risk of musculoskeletal strain from lifting and twisting to move laundry bags. The weighted medical blanket 100 of the invention can be wiped clean with disinfectant in between patient use to reduce risk of infection.

What is claimed is:

1. A reusable medical device comprising:
an inner polymer envelope comprising a first inner layer and a second inner layer surrounded by an outer polymer envelope comprising a first outer surface and a second outer surface;
said inner polymer envelope containing a water absorbing polymer gel; and
said outer polymer envelope is sealed with said inner polymer envelope to create at least a first edge and a second edge.

2. The reusable medical device of claim 1 wherein said polymer gel comprises polyacrylate.

3. The reusable medical device of claim 2 wherein said inner polymer envelope further comprises a volume of water.

4. The reusable medical device of claim 3 wherein said volume of water and said polyacrylate combine to retain a temperature above ambient temperature for more than 20 minutes.

5. The reusable medical device of claim 1 wherein said inner polymer envelope comprises polyvinylchloride.

6. The reusable medical device of claim 5 wherein said outer polymer envelope comprises nylon.

7. The reusable medical device of claim 1 further comprising an insertion sleeve disposed between said first inner layer and said second inner layer and providing access to said inner polymer envelope.

8. The reusable medical device of claim 7 wherein said insertion sleeve further comprises an insert valve configured to prevent flow out of said inner polymer envelope through said insertion sleeve.

9. The reusable medical device of claim 8 wherein said insertion sleeve comprises a proximal end connected to an attachment means of said insert valve and a distal end configured for self-closing.

10. The reusable medical device of claim 8 wherein said insertion sleeve comprises a plurality of baffles to restrict flow out of said sleeve.

11. The device of claim 1 further comprising a plurality of welds between said inner polymer envelope and said outer polymer envelope wherein each weld fixedly binds said inner envelope to said outer envelope to prevent fluid flow at each weld by eliminating space between said first inner layer and said second inner layer at the weld location.

12. The reusable medical device of claim 1 further comprising attachment elements positioned along said first edge.

13. The reusable medical device of claim 12 wherein said attachment elements comprise a plurality of snaps.

14. A method of treating a patient for post-operative shivering comprising the steps of:
Heating a device of claim 4 to a temperature of at least 90 degrees F.; and
Placing said device on a torso of a patient to provide both heat and weight.

15. The method of claim 14 wherein said patient is unconscious.

16. The method of claim 15 wherein said patient under the effect of anesthesia.

17. A reusable medical device for providing heat and weight to a person comprising:
an inner polymer envelope comprising a first inner layer and a second inner layer and surrounded by an outer polymer envelope comprising a first outer surface and a second outer surface;
a super absorbent polymer contained within said inner polymer envelope;
said outer polymer envelope is sealed with said inner polymer envelope to create at least a first edge;
an insertion sleeve disposed between said first inner layer and said second inner layer and providing access to said inner polymer envelope wherein said insertion sleeve is configured to self-close;
a plurality of welds between said inner polymer envelope and said outer polymer envelope wherein each weld fixedly binds said inner envelope to said outer envelope to prevent fluid flow at each weld by eliminating space between said first inner layer and said second inner layer at the weld location; and
at least one attachment element located at said first edge.

18. The device of claim 17 wherein said super absorbent polymer comprises sodium polyacrylate.

19. The device of claim 17 wherein said super absorbent polymer comprises sodium polyacrylate and water.
